(19) United States
(12) Patent Application Publication
(10) Pub. No.: US 2010/0152685 A1
(43) Pub. Date: Jun. 17, 2010

(54) DUAL ASPIRATION LINE FLUIDIC CASSETTE

(76) Inventor: Toh Seng Goh, Ballwin, MO (US)

Correspondence Address: Bausch & Lomb Incorporated, One Bausch & Lomb Place, Rochester, NY, 14604-2701, US

(21) Appl. No.: 12/336,803

(22) Filed: Dec. 17, 2008

(51) Int. Cl. A61M 1/00

(52) U.S. Cl. 604/319

(57) ABSTRACT

An ophthalmic surgical cassette for collecting aspirant fluid and tissue during an ophthalmic surgical procedure. The ophthalmic surgical cassette includes a rigid walled container having an interior volume for collecting aspirant fluid and tissue; and an aspiration manifold coupled to the rigid walled container. The aspiration manifold is connected to an aspiration tube in fluidic communication with the interior volume, a first aspiration line for coupling a surgical handpiece, and a second aspiration line, the second aspiration line being connected to the first aspiration line thereby defining two aspiration paths between the surgical handpiece and the aspiration manifold. The first aspiration line has a first inner diameter, and the second aspiration line has a second inner diameter. The first inner diameter is greater than the second inner diameter such that one of the aspiration paths includes at least one flow characteristic different from the other of the aspiration paths.

### BACKGROUND

1. Field

The present invention is directed to ophthalmic surgical cassettes for ophthalmic surgery. More specifically, the present disclosure is directed towards ophthalmic surgical cassettes with first and second aspiration lines.

2. Description of the Related Art

The statements in this section merely provide background information related to the present disclosure and may not constitute prior art.

Ophthalmic surgical cassettes for use in pump systems during ophthalmic surgical procedures are generally known. Various ophthalmic surgical cassettes commonly include a container for retaining aspirant fluid and tissue retrieved from a patients eye during an ophthalmic surgical procedure. Ophthalmic surgical cassettes generally connect to an aspiration line, which couples the ophthalmic surgical cassette to a surgical handpiece during the ophthalmic surgical procedure.

It is common for an ophthalmic surgical cassette to be placed in a surgery console with a pump for providing aspiration through the aspiration line. The flow rate of the aspiration is affected by the vacuum level of the pump. The pump may generate a low vacuum level for some ophthalmic surgical procedures and a high vacuum level for other ophthalmic surgical procedures. In some ophthalmic surgical procedures, an operator may request a low vacuum level during part of an ophthalmic surgical procedure and request high vacuum level during a different part of said ophthalmic surgical procedure. Therefore, there exists the need for an improved ophthalmic surgical cassette with minimal components and effective performance.

### BRIEF DESCRIPTION OF THE DRAWINGS

The drawing described herein is for illustration purposes only and is not intended to limit the scope of the present disclosure in any way.

The FIGURE is a perspective view of an exemplary embodiment of an ophthalmic surgical cassette according to the present disclosure.

### DETAILED DESCRIPTION OF THE PREFERRED EMBODIMENT

The following description is merely exemplary in nature and is not intended to limit the present disclosure, application, or uses.

According to one embodiment of the present disclosure, an ophthalmic surgical cassette 10 is illustrated in the figure. The ophthalmic surgical cassette 10 includes a rigid walled container 12 having an interior volume for collecting aspirant fluid and/or tissue and an aspiration manifold 14 coupled to the rigid walled container 12. The aspiration manifold 14 is connected to an aspiration tube 16 in fluidic communication with the interior volume, a first aspiration line 18 for coupling a surgical handpiece (not shown), and a second aspiration line 20. The second aspiration line 20 is connected to the first aspiration line 18, thereby defining two aspiration paths A, B between the surgical handpiece and the aspiration manifold 14. The first aspiration line 18 has a first inner diameter. The second aspiration line 20 has a second inner diameter. The first inner diameter is greater than the second inner diameter such that one of the aspiration paths A, B includes at least one flow characteristic different from the other of the aspiration paths A, B.

The first inner diameter of the first aspiration line 18 may be about 1.5 millimeters, and the second inner diameter of the second aspiration line 20 may be about 1.0 millimeters. It should be appreciated that while the inner diameters of the first and second aspirations lines 18, 20 are about 1.5 millimeter and about 1.0 millimeters, respectively, different inner diameters may be employed in other embodiments of the present disclosure, as long as an inner diameter of a first aspiration line is greater than a second inner diameter of a second aspiration line.

In use, the cassette 10 is included in a surgery console (not shown). The surgery console includes multiple plungers, which depress pinch points 22, 24 included in the first and second aspiration lines 18, 20 for separately blocking each of the aspiration paths. The operation of a similar cassette is described in co-pending application U.S. Publication No. 2007/0287959, entitled Ophthalmic Surgical Cassette and System, which is incorporated herein by reference. Thus, an operator may select one of the aspiration lines 18, 20 to aspirate fluid and/or tissue during an ophthalmic surgical procedure, while blocking the other of the aspiration lines 18, 20. Specifically, aspiration may be completed in aspiration path A by blocking the first aspiration line 18 at pinch point 24 or aspiration may be completed in aspiration path B by blocking the second aspiration line 20 at pinch point 22.

In at least one implementation of the cassette 10, a different one of the first and second aspiration lines 18, 20 may be used by an operator depending on a particular vacuum level of a surgery console, in which the cassette 10 is included. For example, an operator may manually select the first aspiration line, which includes a larger inner diameter, to provide an effective flow rate at a lower vacuum level. Conversely, an operator may select the second aspiration line, which includes a smaller inner diameter, to provide an effective flow rate and increased purchase at a higher vacuum level. The operator may toggle between the first and second aspiration lines 18, 20 at various stages of a single ophthalmic surgical procedure and/or between different ophthalmic surgical procedures. In other embodiments, a surgery console may automatically select one of the first and second aspiration lines 18, 20, based on at least a vacuum level provided from a pump included in a surgery console. In this manner, a surgery console including the cassette 10 may provide optimized aspiration performance over a range of vacuum levels provided by the surgery console during one or more ophthalmic surgical procedures. Still other embodiments may be used with peristaltic or flow-based systems.

As shown, the second aspiration line 20 includes a micro-mesh filter 26. The micro-mesh filter 26 may be a StableChamber™ pack sold by the assignee of this invention. The micro-mesh filter 26 may be effective at filtering tissue fragments larger than about 0.5 millimeter. It should be appreciated that a different type of micro-mesh filter having various sizes of mesh for capturing different sized fragments may be included in other embodiments of the present disclosure. A type and/or size of mesh may be different for a phacoemulsification procedure as compared to a different ophthalmic surgical procedure. In addition, other filters or traps may be employed to ensure that line 20 does not become clogged during surgery, as is known in the art.

The cassette 10 includes a generally T-shaped connector 28 connected to the first and second aspiration lines 18, 20. The generally T-shaped connector 28 is further connected to the first aspiration line 18 for coupling to a surgical handpiece. As shown, the second aspiration line 20 including the micro-mesh filter 26 is coupled in parallel with a portion of the first aspiration line 18 between the aspiration manifold 14 and the generally T-shaped connector 28. The generally T-shaped connector 28 is formed separate from the first and second aspiration lines 18, 20 and is included near the micro-mesh filter 26. In other embodiments, a connector between first and second aspiration lines may be positioned otherwise relative to aspiration lines, a rigid walled container and/or a micro-mesh filter.

It should also be appreciated that several different shaped connectors may be employed to connect the first and second aspiration tubes in various embodiments of the present disclosure. For example, a cassette may include a generally Y-shaped connector, a generally F-shaped or a different shaped connector configured to couple at least three aspiration lines. In other embodiments, a connector may be integrally formed with one or more of a first aspiration line and a second aspiration line. In still other embodiments, a connector may be configured to include a micro-mesh filter within an aspiration path associated with at least one aspiration line.

The aspiration manifold 14 is connected to an irrigation manifold 30 for coupling an irrigation source (not shown), e.g., a bottle of balanced salt solution (BSS), via a source irrigation line 32. The irrigation manifold 30 is useful for providing irrigation to the patient's eye via an ophthalmic surgical instrument, e.g., a cannula, through an irrigation line 34. The irrigation manifold 30 may also be in fluidic communication with a reflux tube 36, which is connected to the aspiration manifold 14. Thereby, the ophthalmic surgical cassette 10 may provide irrigation fluid to a patient's eye and to the two aspiration paths A, B during an ophthalmic procedure, rather than requiring a separate irrigation source for each. It should, however, be appreciated that an ophthalmic surgical cassette according to the present disclosure may include a dedicated irrigation source for irrigation of the patient's eye and the aspiration paths.

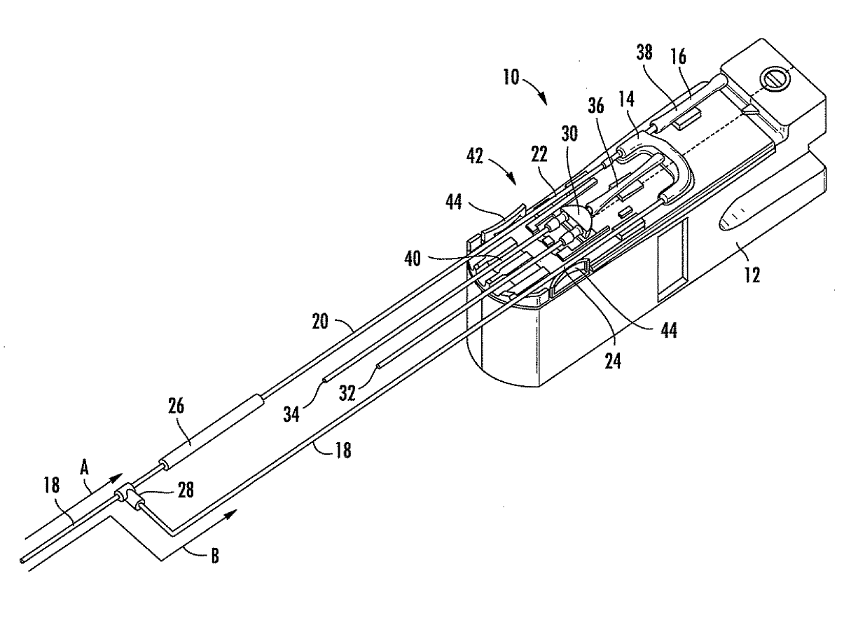

The irrigation manifold 30 may provide irrigation during reflux to ensure effective reflux when multiple refluxes are requested by an operator. While the reflux tube 36 is illustrated as being inline between the aspiration manifold 14 and the irrigation manifold 30, a reflux tube may be offset from between an aspiration manifold and an irrigation manifold in other embodiments of the present disclosure. For example, an aspiration manifold may include a reflux bulb coupled thereto. When reflux is requested from an operator, the pinch point 22 in the second aspiration line 20, a pinch point 38 in the aspiration tube 16, and a pinch point 40 in the irrigation line 34 are depressed. When the reflux tube 36 is actuated, fluid flows in the aspiration path B in a direction opposite of aspiration, thereby potentially clearing an occlusion in the aspiration path B, including the first aspiration line 18. Reflux is not provided through the second aspiration line 20 due to inclusion of the micro-mesh filter 26. While other embodiments of the present disclosure may provide for reflux in two or more aspiration lines, potentially including a filter, reflux is generally limited to aspiration lines without one or more filters included therein. In some embodiments, an operator's request for reflux may cause a surgery console to automatically select an aspiration line without a micro-mesh filter, e.g., the first aspiration line 18 of FIG. 1.

The aspiration manifold 14 is formed on a main manifold 42 that is releasably coupled to the rigid walled container 12. The main manifold 42 is slidable onto the rigid walled container 12, and locked in place by a releasable fastener 44. In use, during an ophthalmic surgical procedure, the rigid walled container 12 may become filled with aspirant fluid and/or tissue such that it is desirable to empty said rigid walled container 12 before continuing in the ophthalmic surgical procedure. Thus, an operator may pinch the releasable fastener 44 and decouple the main manifold 42 from the rigid walled container 12 without disconnecting a surgical handpiece coupled to one or both of the irrigation lines 32, 34 and the two aspirations lines 18, 20, connected to the aspiration manifold 14. It should be appreciated that a different type of releasable fastener may be employed in different embodiments of the present disclosure such that an operator may easily disconnect a main manifold from a rigid walled container during an ophthalmic surgical procedure In still other embodiments, a cassette may includes a main manifold fixedly coupled to the rigid walled container such that disconnecting at least one surgical handpiece may be necessary when the cassette is removed from a surgery console.

Although several aspects of the present disclosure have been described above with reference to ophthalmic surgical cassettes, it should be understood that various aspects of the present disclosure are not limited to ophthalmic surgical cassettes, and can be applied to a variety of other ophthalmic surgical systems, devices and methods.

By implementing any or all of the teachings described above, a number of benefits and advantages can be attained including improved reliability, reduced down time, elimination or reduction of redundant components or systems, avoiding unnecessary or premature replacement of components or systems, and a reduction in overall system and operating costs

We claim:

1. An ophthalmic surgical cassette for collecting aspirant fluid and/or tissue during an ophthalmic surgical procedure, the ophthalmic surgical cassette comprising:
a rigid walled container having an interior volume for collecting aspirant fluid and tissue; and
an aspiration manifold coupled to the rigid walled container, the aspiration manifold connected to an aspiration tube in fluidic communication with the interior volume, a first aspiration line for coupling a surgical handpiece, and a second aspiration line, the second aspiration line being connected to the first aspiration line thereby defining two aspiration paths between the surgical handpiece and the aspiration manifold;
the first aspiration line having a first inner diameter, the second aspiration line having a second inner diameter; the first inner diameter being greater than the second inner diameter such that one of the aspiration paths includes at least one flow characteristic different from the other of the aspiration paths.

2. The invention of claim 1, wherein the second first aspiration line includes a micro-mesh filter.

3. The invention of claim 1, wherein each of the first and second aspiration lines includes a pinch point to select one of the aspiration paths between the surgical handpiece and the aspiration manifold.

4. The invention of claim 1, further comprising a generally T-shaped connector with the first and second aspiration lines connected thereto.

5. The invention of claim 1, wherein the aspiration manifold is further connected to an irrigation manifold for coupling to an irrigation source.

6. The invention of claim 5, further comprising a reflux line connected to the aspiration manifold and in fluidic communication with the irrigation manifold for refluxing the first aspiration line.

7. The invention of claim 1, wherein the aspiration manifold is formed on a main manifold that is releasably coupled to the rigid walled container.

8. An ophthalmic surgical cassette for collecting aspirant fluid and tissue during an ophthalmic surgical procedure, the cassette comprising:
a rigid walled container having an interior volume for collecting aspirant fluid and tissue; and
an aspiration manifold releasably coupled to the rigid walled container, the aspiration manifold connected to a first aspiration line for coupling a surgical handpiece and having a first inner diameter and a second aspiration line having a second inner diameter different than the first inner diameter, the second aspiration line including a micro-mesh filter and coupled in parallel with at least a portion of the first aspiration line thereby providing two aspiration paths between the surgical handpiece and the aspiration manifold.
